(19) United States
(12) Patent Application Publication
(10) Pub. No.: US 2024/0263822 A1
(43) Pub. Date: Aug. 8, 2024

(54) DISPLAY METHOD, TERMINAL DEVICE, DISPLAY SYSTEM, AND STORAGE MEDIUM

(71) Applicant: Panasonic Intellectual Property Management Co., Ltd., Osaka (JP)

(72) Inventors: Taro IIKE, Tokyo (JP); Ippei ODA, Aichi (JP)

(21) Appl. No.: 18/408,435

(22) Filed: Jan. 9, 2024

(30) Foreign Application Priority Data
Feb. 8, 2023 (JP) 2023-017545

(51) Int. Cl. F24F 11/52; G06T 19/00

(52) U.S. Cl. CPC F24F 11/52; G06T 19/00; F24F 2110/50; G06T 2200/24; G06T 2219/004

(57) ABSTRACT

A display method includes an evaluation step of evaluating states of air quality for each type of the air quality on the basis of detection values of an air quality sensor that detects the states of the air quality of target space, and a display step of displaying by a terminal device, a first object indicating the states of the air quality in a spatial image indicating the target space on the basis of evaluation results in the evaluation step, the first object is a combination of a plurality of second objects indicating the states of the air quality for each type of the air quality, and in the display step, the second objects are displayed in colors corresponding to the evaluation results in the evaluation step for each of the second objects while display manner is changed for each of the second objects.

### INCORPORATION BY REFERENCE

The present application claims priority under 35 U.S.C. § 119 to Japanese Patent Application No. 2023-017545 filed on Feb. 8, 2023. The content of the application is incorporated herein by reference in its entirety.

### BACKGROUND OF THE INVENTION

### Field of the Invention

The present disclosure relates to a display method, a terminal device, a display system, and a storage medium.

### Description of the Related Art

Japanese Patent Laid-Open No. 2021-047009 discloses a technique of displaying air quality information indicating air quality in a layout image indicating layout of seats. Further, Japanese Patent Laid-Open No. 2021-047009 discloses an image indicating concentration of CO2 and a room temperature as the air quality information.

The present disclosure provides a display method, a terminal device, a display system, and a storage medium capable of allowing a user to easily grasp states of a plurality of types of air quality and visually confirm an overall state of air quality based on the states of the plurality of types of air quality.

### SUMMARY OF THE INVENTION

A display method in the present disclosure includes an evaluation step of evaluating states of air quality for each type of the air quality on the basis of detection values of an air quality sensor that detects the states of the air quality of target space, and a display step of displaying by a terminal device, a first object indicating the states of the air quality in a spatial image indicating the target space on the basis of evaluation results in the evaluation step, the first object is a combination of a plurality of second objects indicating the states of the air quality for each type of the air quality, and in the display step, the second objects are displayed in colors corresponding to the evaluation results in the evaluation step for each of the second objects while display manner is changed for each of the second objects.

Further, a terminal device in the present disclosure includes a display control unit that displays a first object indicating states of air quality in a spatial image indicating target space using a display unit on the basis of evaluation results by an evaluation unit that evaluates the states of the air quality for each type of the air quality on the basis of detection values of an air quality sensor that detects the states of the air quality of the target space, the first object is a combination of a plurality of second objects indicating the states of the air quality for each type of the air quality, and the display control unit displays the second objects in colors corresponding to the evaluation results by the evaluation unit for each of the second objects while changing display manner for each of the second objects.

Further, a display system in the present disclosure includes a display unit, an evaluation unit that evaluates states of air quality for each type of the air quality on the basis of detection values of an air quality sensor that detects the states of the air quality of target space, and a display control unit that displays a first object indicating the states of the air quality in a spatial image indicating the target space using the display unit on the basis of evaluation results by the evaluation unit, the first object is a combination of a plurality of second objects indicating the states of the air quality for each type of the air quality, and the display control unit displays the second objects in colors corresponding to the evaluation results by the evaluation unit for each of the second objects while changing display manner for each of the second objects.

Further, a storage medium storing a program in the present disclosure causes a processor of a terminal device to function as a display control unit that displays a first object indicating states of air quality in a spatial image indicating target space using a display unit on the basis of evaluation results by an evaluation unit that evaluates the states of the air quality for each type of the air quality on the basis of detection values of an air quality sensor that detects the states of the air quality of the target space, the first object is a combination of a plurality of second objects indicating the states of the air quality for each type of the air quality, and the display control unit displays the second objects in colors corresponding to the evaluation results by the evaluation unit for each of the second objects while changing display manner for each of the second objects.

A display method, a terminal device, a display system, and a storage medium in the present disclosure allow a user to easily grasp impression of an overall state of air quality based on states of a plurality of types of air quality by displaying a plurality of second objects in colors corresponding to evaluation results. Further, the display method, the terminal device, the display system, and the storage medium in the present disclosure can clearly specify that a first object is not part of target space indicated by a spatial image by displaying the second objects while changing display manner. It is therefore possible to allow the user to easily grasp that the first object is an object indicating the states of the air quality. Thus, it is possible to allow the user to easily grasp the states of the plurality of types of air quality and visually confirm an overall state of the air quality based on the states of the plurality of types of air quality.

### BRIEF DESCRIPTION OF THE DRAWINGS

FIG. 1 is a view illustrating a configuration of a display system in a first embodiment;

FIG. 2 is a view schematically illustrating target space viewed from above in the first embodiment;

FIG. 3 is a view illustrating configurations of a terminal device and a server device in the first embodiment;

FIG. 4 is a view illustrating one example of a first management DB and a second management DB in the first embodiment;

FIG. 5 is a view illustrating one example of an application UI in the first embodiment;

FIG. 6 is a view illustrating one example of a first region in the first embodiment;

FIG. 7 is a view illustrating one example of a first object in the first embodiment;

FIG. 8 is a view illustrating one example of an alert in the first embodiment;

FIG. 9 is a view for explaining a third object in the first embodiment;

FIG. 10 is a view illustrating one example of change in display manner of a second object in the first embodiment;

FIG. 11 is a view illustrating one example of the application UI in the first embodiment;

FIG. 12 is a flowchart indicating operation of the display system in the first embodiment;

FIG. 13 is a flowchart indicating operation of the display system in the first embodiment;

FIG. 14 is a flowchart indicating operation of the terminal device in the first embodiment;

FIG. 15 is a flowchart indicating operation of the terminal device in the first embodiment; and

FIG. 16 is a view illustrating one example of an application UI in a second embodiment.

### DETAILED DESCRIPTION OF THE PREFERRED EMBODIMENTS

(Knowledge, or the Like, that Becomes Basis of the Present Disclosure)

At the time when the inventors conceived of the present disclosure, there was a technique of displaying information indicating air quality as disclosed in Japanese Patent Laid-Open No. 2021-047009. With Japanese Patent Laid-Open No. 2021-047009, whether or not a circular image is an image constituting part of layout cannot be grasped at first glance, and it is difficult to grasp whether or not the circular image is an image indicating states of air quality. Further, the inventors have found problems that with Japanese Patent Laid-Open No. 2021-047009, states of a plurality of types of air quality are indicated with one circular image, and thus, it is difficult to make a user intuitively grasp each state of the plurality of types of air quality and it is difficult to make the user easily grasp impression of an overall state of air quality based on the states of the plurality of types of air quality, and have achieved subject of the present disclosure to solve the problems.

The present disclosure therefore provides a display method, a terminal device, a display system, and a storage medium capable of allowing a user to easily grasp states of a plurality of types of air quality and visually confirm an overall state of air quality based on the states of the plurality of types of air quality.

Embodiments will be described in detail below with reference to the drawings. However, there is a case where detailed description more than necessary will be omitted. For example, there is a case where detailed description regarding well known matters or redundant description regarding substantially the same components will be omitted.

Note that the accompanying drawings and the description below are provided to allow a person skilled in the art to sufficiently understand the present disclosure and are not intended to limit the subject matter recited in the claims.

### First Embodiment

### [1-1. Configuration]

### [1-1-1. Configuration of Display System]

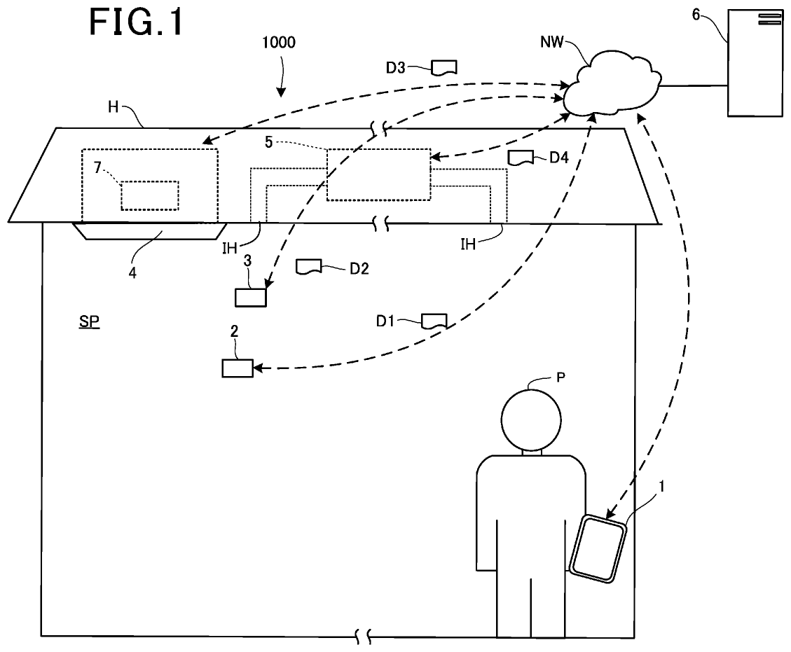

FIG. 1 is a view illustrating a configuration of a display system 1000.

The display system 1000 is a system in which a terminal device 1 can display states of air quality of target space SP. The target space SP is space in which an air quality sensor 2 detects air quality and is internal space of a building H, that is, indoor space.

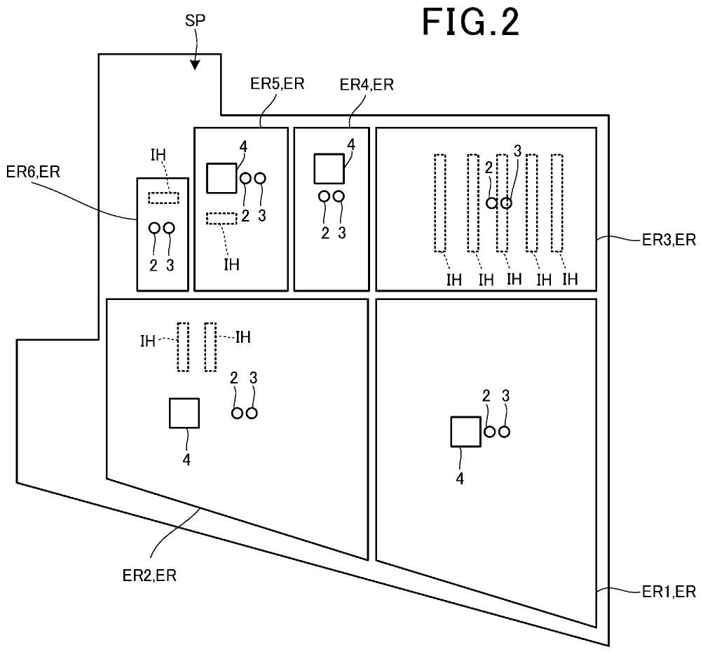

FIG. 2 is a view schematically illustrating the target space SP viewed from above.

The target space SP is divided into six areas ER of a first area ER1, a second area ER2, a third area ER3, a fourth area ER4, a fifth area ER5, and a sixth area ER6.

In each of the first area ER1 to the sixth area ER6, the air quality sensor 2 and a wind speed sensor 3 are provided. The air quality sensor 2 and the wind speed sensor 3 will be described later.

Further, in each of the first area ER1, the second area ER2, the fourth area ER4, and the fifth area ER5, an indoor unit 4 is provided.

Further, in the second area ER2, the third area ER3, the fifth area ER5, and the sixth area ER6, outlets IH that discharge hypochlorous acid generated by a first air purification device 5 are provided.

Returning to description of FIG. 1, the display system 1000 includes the air quality sensor 2. The display system 1000 of the present embodiment includes six air quality sensors 2.

The air quality sensor 2, which is a sensor that detects air quality, is provided within the target space SP. The air quality sensor 2 of the present embodiment detects a temperature, humidity, concentration of CO2 (hereinafter, referred to as “CO2 concentration”), concentration of PM2.5 (hereinafter, referred to as “PM2.5 concentration”), concentration of microorganisms (for example, mold and virus) (hereinafter, referred to as “microorganism concentration”), and odor as air quality. Thus, the air quality sensor 2 includes a temperature sensor, a humidity sensor, a CO2 sensor that detects CO2 concentration, a PM2.5 sensor that detects PM2.5 concentration, a microorganism sensor that detects microorganism concentration, and an odor sensor that detects odor. Note that the CO2 sensor is, for example, a sensor employing a non-dispersive infrared absorption method. The PM2.5 sensor is, for example, an optical sensor including a light emission unit and a light reception unit. The microorganism sensor is, for example, a respiration measurement or electrode active material measurement sensor. The odor sensor is, for example, a sensor in which a sensor element is constituted with a semiconductor.

The PM2.5 and microorganisms are one example of an “air-polluting substance”.

If the air quality sensor 2 detects six types of air quality with a predetermined period (for example, with a period of five minutes), the air quality sensor 2 transmits first detection data D1 to a server device 6 every time the air quality is detected. The first detection data D1 includes detection values of the six types of air quality. A first sensor ID (identification) that identifies the air quality sensor 2 is added to the first detection data D1. Note that the air quality sensor 2 may communicate with the server device 6 connected to a network NW via a local network constructed in the building H or may communicate with the server device 6 without passing through the local network. The network NW includes the Internet, a telephone network and other communication networks.

The display system 1000 includes the wind speed sensor 3. The display system 1000 of the present embodiment includes six wind speed sensors 3.

The wind speed sensor 3, which is a sensor that detects wind speed, is provided within the target space SP. The wind speed sensor 3 detects wind speed, for example, by temperature change in a heated element. The wind speed sensor 3 is provided around the air quality sensor 2. For example, the wind speed sensor 3 is provided at a position less than a predetermined distance (for example, 20 cm) from the air quality sensor 2 provided in the same area ER. The wind speed sensor 3 detects wind speed with a predetermined period (for example, with a period of five minutes) and transmits second detection data D2 to the server device 6 every time the wind speed is detected. The second detection data D2 includes a detection value of the wind speed sensor 3. A second sensor ID that identifies the wind speed sensor 3 is added to the second detection data D2 to be transmitted to the server device 6. Note that the wind speed sensor 3 may communicate with the server device 6 connected to the network NW via the local network constructed in the building H or may communicate with the server device 6 without passing through the local network.

The display system 1000 includes the indoor unit 4. The display system 1000 of the present embodiment includes four indoor units 4.

The indoor unit 4 constitutes an air conditioning device with an exterior unit (not illustrated) and performs air conditioning of the target space SP. While a ceiling cassette indoor unit is illustrated as the indoor unit 4 of the present embodiment, a type of the indoor unit 4 may be other types such as a wall-mounted type and a ceiling hanging type. The indoor unit 4 communicates with the server device 6.

The indoor unit 4 includes a second air purification device 7.

The second air purification device 7 generates nanosized particulate ions by applying a high voltage to water in the air and discharges the generated particulate ions to the target space SP via the outlet of the indoor unit 4. By this means, the second air purification device 7 purifies air within the target space SP by oxidation power of OH radical included in the particulate ions. Note that the particulate ions to be discharged by the second air purification device 7 have a sterilization effect, a deodorizing effect, and the like.

The indoor unit 4 transmits first status data D3 to the server device 6 with a predetermined period. In the first status data D3, which is data indicating a state of the indoor unit 4, whether or not the second air purification device 7 is operating, an air volume to be supplied by the indoor unit 4, a wind direction of the indoor unit 4 are recorded.

An indoor unit ID that identifies the indoor unit 4 is added to the first status data D3. Note that the indoor unit 4 may communicate with the server device 6 connected to the network NW via the local network constructed in the building H or may communicate with the server device 6 without passing through the local network.

The display system 1000 includes the first air purification device 5. The display system 1000 of the present embodiment includes one first air purification device 5.

The first air purification device 5 discharges air including hypochlorous acid by making sucked air pass through a filter impregnated with a hypochlorite solution. The air to be discharged by the first air purification device 5 has a sterilization effect, a deodorizing effect, and the like. The first air purification device 5 of the present embodiment is a ceiling-embedded device and discharges hypochlorous acid from the outlet IH through a duct. Note that a type of the first air purification device 5 is not limited to a ceiling-embedded type and may be other types such as a floor-standing type standing within the target space SP.

Hereinafter, in a case where hypochlorous acid to be discharged by the first air purification device 5 and particulate ions to be discharged by the second air purification device 7 are not distinguished from each other, they will be referred to as an “air-purifying substance”.

The first air purification device 5 transmits second status data D4 to the server device 6 with a predetermined period. In the second status data D4, which is data indicating a state of the first air purification device 5, whether or not hypochlorous acid is being discharged and an air volume to be supplied by the first air purification device 5 are recorded. A device ID that identifies the first air purification device 5 is added to the second status data D4. Note that the first air purification device 5 may communicate with the server device 6 connected to the network NW via the local network constructed in the building H or may communicate with the server device 6 without passing through the local network.

The display system 1000 includes the terminal device 1.

In the present embodiment, a device including a touch panel 12 is exemplified as the terminal device 1. Note that examples of the terminal device 1 of the present embodiment can include a smartphone and a tablet computer. An application program for displaying states of air of the target space SP is installed in the terminal device 1. In the following description, this application program will be referred to as a “display application 111”. The terminal device 1 communicates with the server device 6 by a function of the display application 111.

The display application 111 is one example of a “program”.

The display system 1000 includes the server device 6.

The server device 6 is a device that performs information processing using the air quality sensor 2, the wind speed sensor 3, the indoor unit 4, the first air purification device 5, and the terminal device 1 as clients. The server device 6 is connected to the network NW and communicates with these clients. Note that while the server device 6 is expressed with one block in the respective drawings, this does not necessarily mean that the server device 6 is constituted with a single device.

Configurations of the terminal device 1 and the server device 6 will be described next.

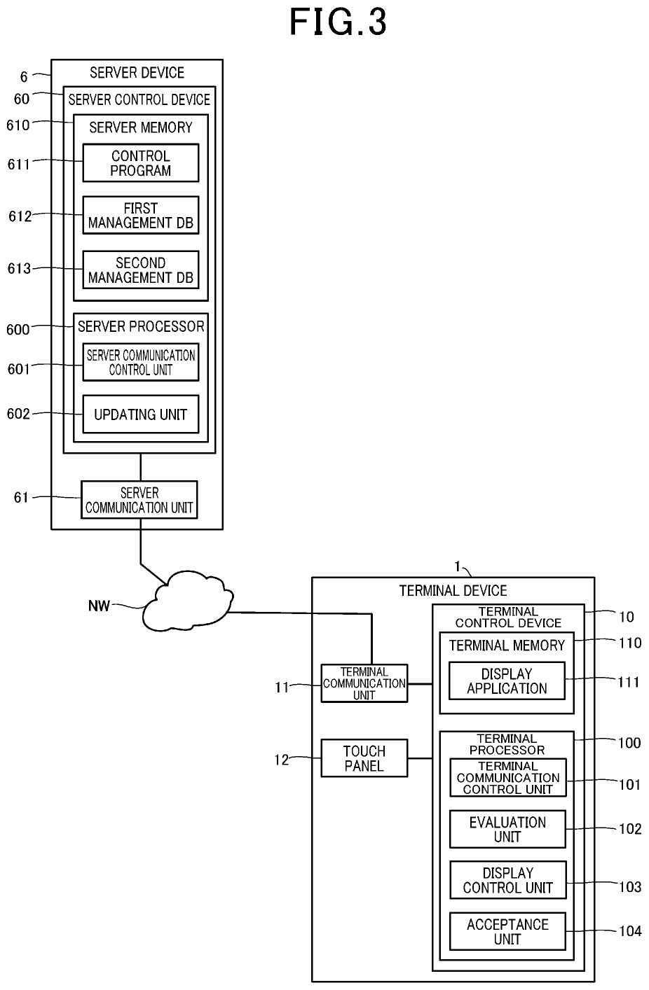

FIG. 3 is a view illustrating the configurations of the terminal device 1 and the server device 6.

### [1-2. Configuration of Server Device]

The configuration of the server device 6 will be described next.

The server device 6 includes a server control device 60 and a server communication unit 61.

The server control device 60 is a control device that controls respective units of the server device 6. The server control device 60 includes a server processor 600 that is a processor such as a central processing unit (CPU) and a micro processor unit (MPU), a server memory 610, and an interface circuit for connecting other devices and sensors and controls the respective units of the server device 6.

The server memory 610 is a memory (storage medium) that stores a program and data. The server memory 610 stores a control program 611, a first management database (DB) 612, a second management DB 613 and data to be processed by the server processor 600. The server memory 610 has a non-volatile storage area. Further, the server memory 610 may include a volatile storage area and may constitute a work area of the server processor 600. The server memory 610 is constituted with, for example, a ROM and a RAM.

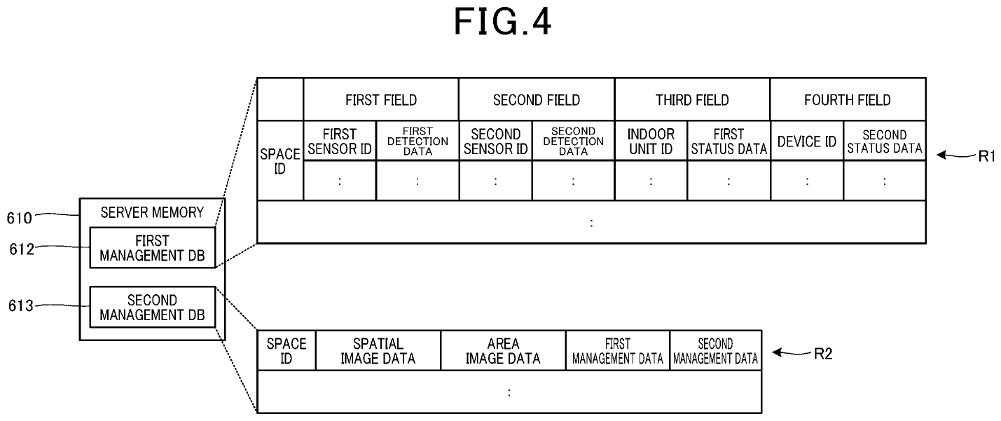

FIG. 4 is a view illustrating one example of the first management DB 612 and the second management DB 613.

The first management DB 612 has a record R1 for each target space SP.

The record R1 has a first field, a second field, a third field, and a fourth field. In the record R1, a space ID is associated with these fields.

In the first field, a combination of the first sensor ID and the first detection data D1 is recorded for each air quality sensor 2 provided in the target space SP.

In the second field, a combination of the second sensor ID and the second detection data D2 is recorded for each wind speed sensor 3 provided in the target space SP.

In the third field, a combination of the indoor unit ID and the first status data D3 is recorded for each indoor unit 4 that performs air conditioning of the target space SP.

In the fourth field, a combination of the device ID and the second status data D4 is recorded for each first air purification device 5 that supplies hypochlorous acid to the target space SP.

The second management DB 613 has a record R2 for each target space SP.

The record R2 has a space ID, spatial image data, area image data, management data, and position data.

The spatial image data is image data indicating a spatial image G1. The spatial image G1 will be described later.

The area image data is image data indicating an area image G2. The area image G2 will be described later.

The first management data is data that manages which indoor unit 4 is provided in which area ER, which air quality sensor 2 is provided in which area ER, and which wind speed sensor 3 is provided in which area ER. More specifically, IDs (the first sensor ID, the second sensor ID and the indoor unit ID) of equipment provided in the area ER are recorded in the management data for each area ER.

In the second management data, a combination of a position in the spatial image G1 corresponding to the position of the air quality sensor 2 in the target space SP, the first sensor ID and the second sensor ID is recorded for each air quality sensor 2. Further, in the second management data, a combination of a position in the spatial image G1 corresponding to the position of the indoor unit 4 in the target space SP and the indoor unit ID is recorded for each indoor unit 4. Further, in the second management data, a position in the spatial image G1 corresponding to a position of the outlet IH in the target space SP is recorded for each outlet IH.

The server communication unit 61, which includes communication hardware such as a communication circuit, communicates with the air quality sensor 2, the wind speed sensor 3, the indoor unit 4, the first air purification device 5, and the terminal device 1 in accordance with control by the server control device 60. Communication standards of the server communication unit 61 may be either wireless communication standards or wired communication standards.

The server processor 600 functions as a server communication control unit 601 and an updating unit 602 by reading out and executing the control program 611 stored in the server memory 610.

The server communication control unit 601 communicates with the air quality sensor 2, the indoor unit 4, the first air purification device 5, and the terminal device 1 via the server communication unit 61.

The updating unit 602 updates content of the record R1.

In a case where the server communication control unit 601 receives the first detection data D1, the updating unit 602 updates the first detection data D1 associated with the first sensor ID added to the received first detection data D1 to the received first detection data D1.

In a case where the server communication control unit 601 receives the second detection data D2, the updating unit 602 updates the second detection data D2 associated with the second sensor ID added to the received second detection data D2 to the received second detection data D2.

In a case where the server communication control unit 601 receives the first status data D3, the updating unit 602 updates the first status data D3 associated with the indoor unit ID added to the received first status data D3 to the received first status data D3.

In a case where the server communication control unit 601 receives the second status data D4, the updating unit 602 updates the second status data D4 associated with the device ID added to the received second status data D4 to the received second status data D4.

### [1-3. Configuration of Terminal Device]

The configuration of the terminal device 1 will be described next.

The terminal device 1 includes a terminal control device 10, a terminal communication unit 11, and a touch panel 12.

The terminal control device 10 is a control device that controls respective units of the terminal device 1. The terminal control device 10 includes a terminal processor 100 that is a processor such as a CPU and an MPU, a terminal memory 110, and an interface circuit for connecting other devices and sensors and controls the respective units of the terminal device 1.

The terminal processor 100 corresponds to one example of a “processor”. The touch panel 12 corresponds to one example of a “display unit”.

The terminal memory 110 is a memory (storage medium) that stores a program and data. The terminal memory 110 stores a display application 111, and data to be processed by the terminal processor 100. The terminal memory 110 has a non-volatile storage area. Further, the terminal memory 110 may include a volatile storage area and constitute a work area of the terminal processor 100. The terminal memory 110 is, for example, constituted with a ROM and a RAM.

The terminal communication unit 11, which includes communication hardware such as a communication circuit, communicates with the server device 6 connected to the network NW in accordance with control by the terminal control device 10. Communication standards of the terminal communication unit 11 may be either wireless communication standards or wired communication standards.

The touch panel 12 includes a display panel, and a touch sensor provided in an overlapped manner on the display panel or provided integrally with the display panel. The display panel displays various kinds of information under control by the terminal control device 10.

The touch sensor detects touch operation and outputs a detection signal to the terminal control device 10. The terminal control device 10 executes processing corresponding to the touch operation on the basis of input from the touch sensor.

The terminal processor 100 functions as a terminal communication control unit 101, an evaluation unit 102, a display control unit 103, and an acceptance unit 104 by reading out and executing the display application 111 stored in the terminal memory 110.

The terminal communication control unit 101 communicates with the server device 6 via the terminal communication unit 11.

The evaluation unit 102 evaluates states of air quality detected by the air quality sensor 2 on the basis of detection values of the air quality sensor 2. The evaluation unit 102 evaluates degrees of goodness or badness of the states of the air quality for each type of air quality. The evaluation unit 102 evaluates the degrees of goodness or badness of the states of the air quality in a plurality of stages. For example, the evaluation unit 102 evaluates the degrees in five stages of “very poor”, “poor”, “fair”, “best”, and “very best”.

Note that in a case of this example, the evaluation results of the evaluation unit 102 indicate higher evaluation, that is, better states of the air quality in order of “very poor”, “poor”, “fair”, “best”, and “very best”. Note that this example does not limit the stages of evaluation by the evaluation unit 102 to five stages. The stages of the evaluation by the evaluation unit 102 may four stages or less or six stages or more.

An evaluation index of the evaluation unit 102 differs for each type of air quality. In other words, the evaluation unit 102 evaluates the detection values of the air quality sensor 2 for each type of air quality using an evaluation index determined for each type of air quality. While units of the detection values differ depending on types of air quality, the evaluation unit 102 can perform appropriate evaluation without depending on types of air quality by using the evaluation index determined for each type of air quality. The evaluation unit 102 outputs the evaluation results to the display control unit 103.

The display control unit 103 displays a user interface that displays the states of air quality of the target space SP using the touch panel 12. In the following description, this user interface will be referred to as an “application UI 120”.

The acceptance unit 104 accepts various kinds of operation from a user P via the application UI 120.

### [1-4. Configuration of Application UI]

The application UI 120 will be described next.

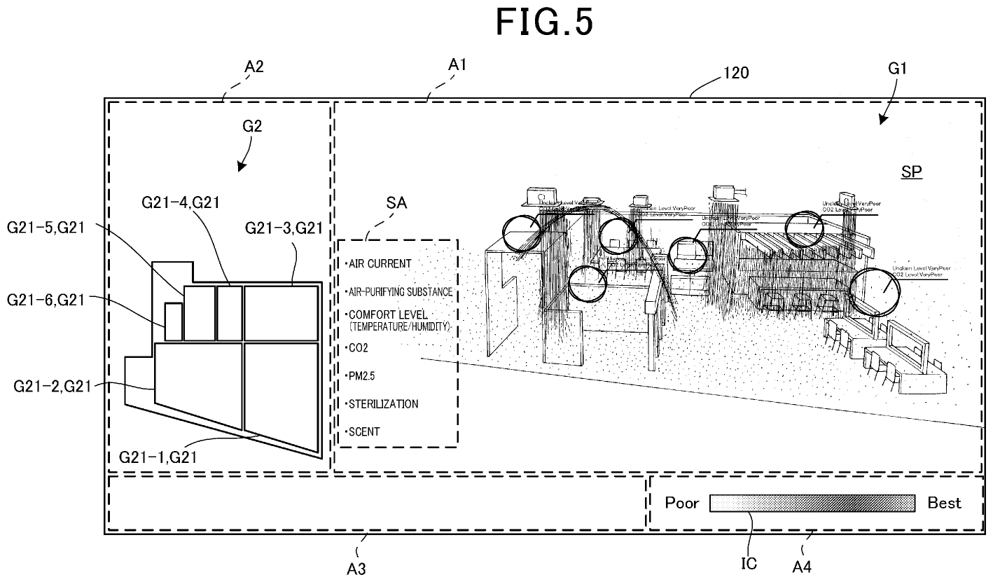

FIG. 5 is a view illustrating one example of the application UI 120.

The application UI 120 has a first region A1, a second region A2, a third region A3, and a fourth region A4. In FIG. 5, the first region A1 and the second region A2 are arranged in a horizontal direction, and the third region A3 and the fourth region A4 are arranged in the horizontal direction below the first region A1 and the second region A2. Note that a manner of arrangement of the first region A1 to the fourth region A4 is not limited to that illustrated in FIG. 5.

The first region A1 is a region for displaying the states of the air quality of the target space SP.

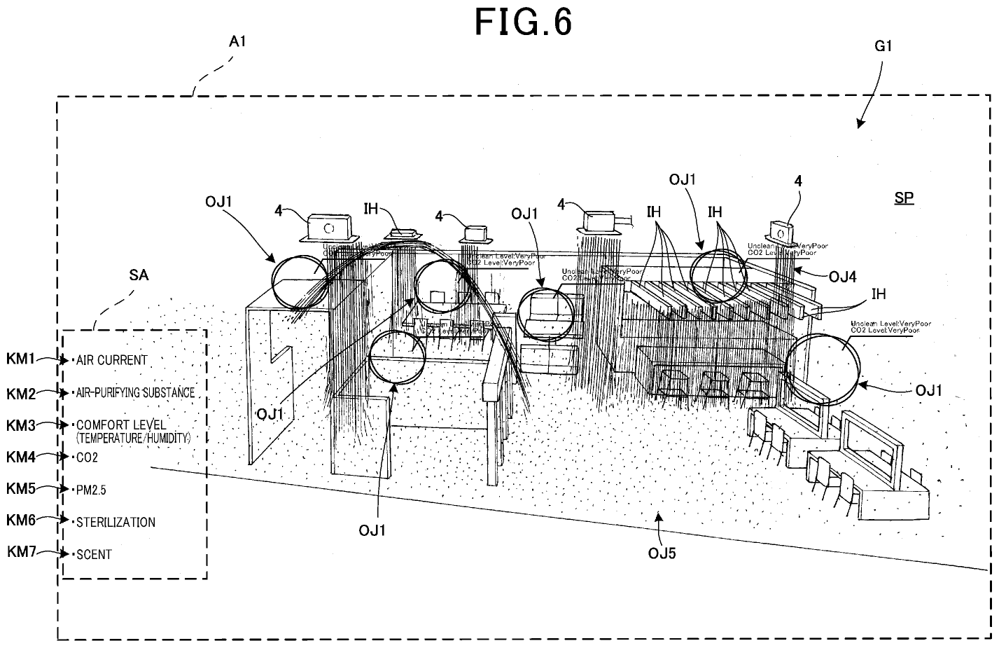

The first region A1 will be described in detail with reference to FIG. 6.

FIG. 6 is a view illustrating one example of the first region A1.

The first region A1 displays the spatial image G1. The spatial image G1 is a top-down image of the target space SP viewed from a predetermined point of view and three-dimensionally indicates the target space SP. The target space SP indicated by the spatial image G1 mimics an actual target space SP. Thus, layout of the target space SP indicated by the spatial image G1 matches actual layout. Further, in the target space SP indicated by the spatial image G1, at least the indoor unit 4 and the outlet IH are indicated at positions that are the same as positions where they are actually provided.

In the first region A1, a position of the point of view from which the target space SP is overlooked can be changed. More specifically, in the first region A1, the position of a line of sight from which the target space SP is overlooked can be changed by being rotated around the center of a horizontal plane of the target space SP in a range of 360°.

The acceptance unit 104 accepts swipe operation or flick operation in the first region A1.

Note that the swipe operation refers to operation in which a position where the finger, or the like, of the user P makes contact with the touch panel 12 moves in a state where the finger is in contact with the touch panel 12. The flick operation refers to operation in which the finger, or the like, of the user P is moved away from the touch panel 12 in a manner of flicking from a state where the finger, or the like, of the user P is in contact with the touch panel 12. If the acceptance unit 104 accepts the swipe operation or the flick operation in the horizontal direction in FIG. 6, in the first region A1, the spatial image G1 whose position of the point of view from which the target space SP is overlooked is changed in accordance with an operation amount is displayed.

The first region A1 displays a first object OJ1 in an overlapped manner on the spatial image G1.

The first object OJ1 is an object indicating states of a plurality of types of air quality and is an image. The first object OJ1 is displayed at one of positions in the spatial image G1 corresponding to the position where the air quality sensor 2 is provided. In the present embodiment, six air quality sensors 2 are provided in the target space SP. Thus, in the spatial image G1, the first objects OJ1 are displayed at respective positions corresponding to the six air quality sensors 2. In other words, in the spatial image G1 of the present embodiment, six first objects OJ1 are displayed at different positions. Note that while the first objects OJ1 are not colored in the respective drawings to facilitate understanding, actual first objects OJ1 are colored.

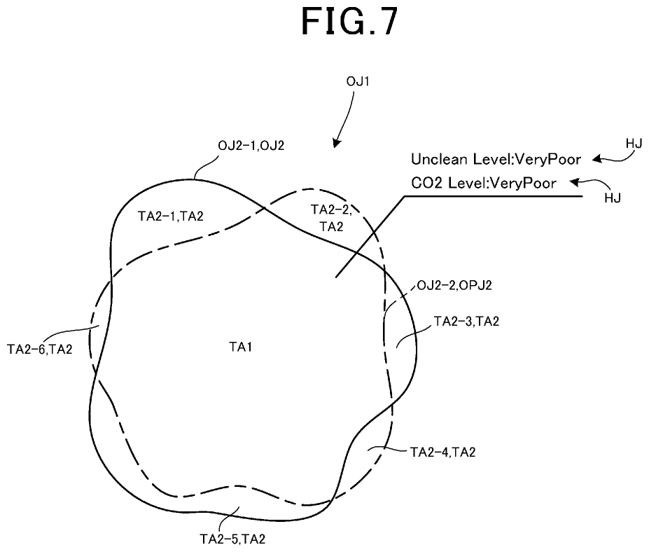

FIG. 7 is a view illustrating one example of the first object OJ1.

The first object OJ1 includes one or a plurality of second objects OJ2. In other words, in a case where the first object OJ1 includes a plurality of second objects OJ2, the first object OJ1 is an object that is a combination of the plurality of second objects OJ2. One second object OJ2 is an object regarding one type of air quality and indicates a state of the air quality. The second object OJ2 is an image. In a case where the first object OJ1 includes a plurality of second objects OJ2, the plurality of second objects OJ2 are displayed in the first region A1 in a state where at least part of the second objects OJ2 overlaps with each other.

Colors of the second object OJ2 are colors corresponding to the evaluation results by the evaluation unit 102. As described above, the evaluation unit 102 evaluates degrees of goodness or badness of the states of the air quality detected by the air quality sensor 2 for each type of air quality. The second objects OJ2 are displayed in colors in accordance with color phases from red to blue. The color of the second object OJ2 becomes closer to red as the evaluation result by the evaluation unit 102 is lower evaluation. On the other hand, the color of the second object OJ2 becomes closer to blue as the evaluation by the evaluation unit 102 is higher evaluation.

As described above, in a case where the first object OJ1 includes a plurality of second objects OJ2, the first object OJ1 is displayed in the first region A1 in a state where at least part of the second objects OJ2 overlaps with each other. In the first object OJ1, a color of an overlapping region TAL in which the second objects OJ2 overlap with each other is different from a color of a non-overlapping region TA2 in which the second objects OJ2 do not overlap with each other. The color of the overlapping region TAL is set to a mixed color of the overlapping second objects OJ2. The color of the non-overlapping region TA2 is set to a color of the corresponding second object OJ2.

In FIG. 7, a case is exemplified where the first object OJ1 includes a second object OJ2-1 and a second object OJ2-2. Thus, in FIG. 7, the color of the overlapping region TAL is set to a mixed color of the second objects OJ2-1 and OJ2-2. Further, the color of non-overlapping regions TA2-1, TA2-3, and TA2-5 in FIG. 7 is set to the color of the second object OJ2-1. Further, in FIG. 7, the color of non-overlapping regions TA2-2, TA2-4, and TA2-6 is set to the color of the second object OJ2-2.

The first object OJ1 includes the second objects OJ2 of the number corresponding to the number of items selected in a selection region SA of the first region A1. The selection region SA is a region for selecting objects to be displayed in the first region A1 and includes the following seven items.

First item: item for displaying an object regarding an air current

Second item: item for displaying an object regarding an air-purifying substance

Third item: item for displaying an object regarding humidity and a temperature of the target space SP

Fourth item: item for displaying an object regarding CO2 concentration of the target space SP

Fifth item: item for displaying an object regarding PM2.5 concentration of the target space SP

Sixth item: item for displaying an object regarding microorganism concentration of the target space SP Seventh item: item for displaying an object regarding odor of the target space SP

In a case where none of the third to the seventh items is selected in the selection region SA, the first object OJ1 is not displayed in the spatial image G1. On the other hand, in a case where one or a plurality of items are selected from the third to the seventh items in the selection region SA, the first object OJ1 including the second objects OJ2 corresponding to the selected items is displayed in the spatial image G1. For example, in a case where five items of the third to the seventh items are selected, the first object OJ1 including five second objects OJ2 corresponding to the five items is displayed in the spatial image G1.

Evaluation information HJ is displayed in the first object OJ1 for each of the second objects OJ2 of the first object OJ1. The evaluation information HJ is information in which names of items are associated with evaluation results by the evaluation unit 102.

In the evaluation information HJ of the second object OJ2 corresponding to the third item, for example, evaluation results regarding a temperature and humidity detected by the air quality sensor 2 are associated with the name of the item of “Comfort Level”.

In the evaluation information HJ of the second object OJ2 corresponding to the fourth item, for example, an evaluation result regarding CO2 concentration detected by the air quality sensor 2 is associated with the name of the item of “CO2 Level”.

In the evaluation information HJ of the second object OJ2 corresponding to the fifth item, for example, an evaluation result of PM2.5 concentration detected by the air quality sensor 2 is associated with the name of the item of “Unclean level” using alphabetical notation.

In the evaluation information HJ of the second object OJ2 corresponding to the sixth item, for example, an evaluation result regarding microorganism concentration detected by the air quality sensor 2 is associated with the name of the item of “Sanitixe Level”.

In the evaluation information HJ of the second object OJ2 corresponding to the seventh item, for example, an evaluation result regarding odor detected by the air quality sensor 2 is associated with the name of the item of “Scent Level”.

In a case where concerning the air quality indicated by the second object OJ2 of the first object OJ1, the detection value of the air quality sensor 2 is equal to or greater than a threshold, an alert AR is displayed in association with the first object OJ1. This threshold is different for each type of air quality and is determined in advance through test or simulation performed in advance on the basis of a viewpoint of necessity to display the alert AR.

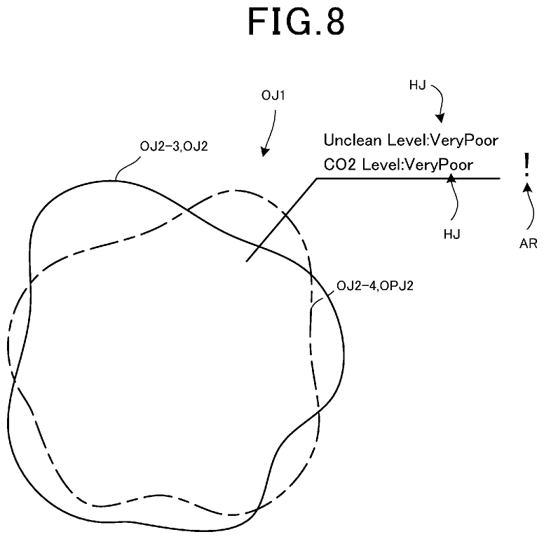

FIG. 8 is a view illustrating one example of the alert AR.

While in FIG. 8, an exclamation mark is used as the alert AR, the alert AR may be other symbols, characters, character strings, images, or the like.

FIG. 8 indicates that the CO2 concentration is equal to or greater than the threshold.

The second object OJ2 will be described in detail. The second object OJ2 of each of the third item and the fourth item includes a third object OJ3 inside.

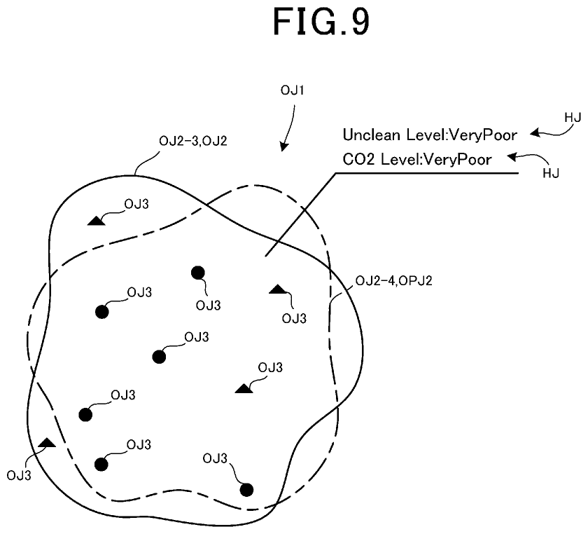

FIG. 9 is a view for explaining the third object OJ3.

The third object OJ3 is an object indicating an air-polluting substance that pollutes air of the target space SP and is an image. The third object OJ3 included in the second object OJ2 of the third item is an object indicating PM2.5. The third object OJ3 included in the second object OJ2 of the fourth item is an object indicating microorganisms. At least one of shapes or colors of the third objects OJ3 are different in accordance with types of the items. FIG. 9 illustrates an example of a case where shapes of the third objects OJ3 are different in accordance with types of the items.

Note that the shapes and the colors of the third objects OJ3 may be the same regardless of types of the items.

In FIG. 9, the second object OJ2 corresponding to the third item is set as a second object OJ2-3, and a shape of the third object OJ3 of the second object OJ2-3 is made a circular shape.

Further, in FIG. 9, the second object OJ2 corresponding to the fourth item is set as a second object OJ2-4, and a shape of the third object OJ3 of the second object OJ2-4 is made a triangular shape.

The number of the third objects OJ3 included in the second object OJ2 corresponds to the states of the air quality, that is, the detection values of the air quality sensor 2. In other words, the number of the third objects OJ3 included in the second object OJ2 of the third item increases as the PM2.5 concentration detected by the air quality sensor 2 is higher. In a similar manner, the number of the third objects OJ3 included in the second object OJ2 of the fourth item increases as the microorganism concentration detected by the air quality sensor 2 is higher.

In a case where the first object OJ1 includes two second objects OJ2 of the third item and the fourth item, a total number of the third objects OJ3 of the first object OJ1 is the number obtained by adding the number of the third objects OJ3 of the second object OJ2 of the third item and the number of the third objects OJ3 of the second object OJ2 of the fourth item.

The second objects OJ2 are displayed in a manner that outer edges OE that are closed curves deform with time. More specifically, the second objects OJ2 are displayed with the outer edges OE wobbling.

Display with the outer edges OE wobbling means display with the outer edges OE rippling. In other words, display with the outer edges OE wobbling means display in a manner that positions of concavo-convex portions in the outer edges OE change with time.

Note that the shapes of the second objects OJ2 are the same regardless of types of air quality in a case where the outer edges OE are not made to wobble.

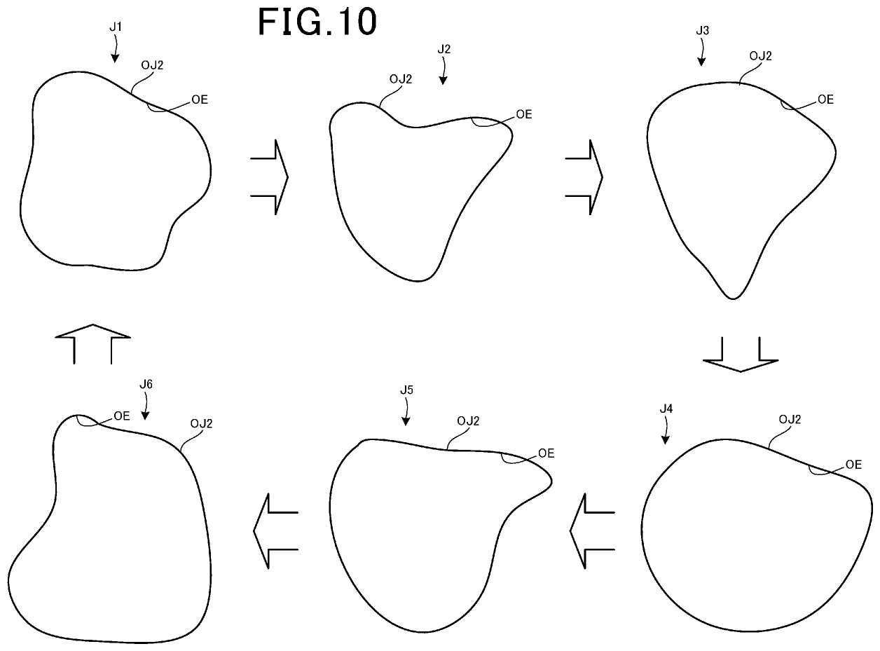

FIG. 10 is a view illustrating one example of change in display manner of the second object OJ2.

In display of the second object OJ2, the shape of the outer edge OE of the second object OJ2 returns to the original shape with a predetermined period. FIG. 10 illustrates an example of a case where the shape returns to a shape J1 by way of five shapes of shapes J2, J3, J4, J5, and J6. Note that the shapes by way of which the outer edge OE of the second object OJ2 returns to the original shape are not limited to six shapes and may be equal to or less than five shapes or more shapes.

The shape of the outer edge OE of the second object OJ2 returns to the original shape by way of different shapes in accordance with types of the items selectable in the selection region SA. By this means, in a case where the first object OJ1 includes a plurality of second objects OJ2, it is possible to reduce a frequency of the second objects OJ2 completely overlapping with each other. Note that while the shape of the outer edge OE of the second object OJ2 may return to the original shape by way of the same shapes regardless of types of the items selectable in the selection region SA, in this case, timings at which the shape returns to the original shape are preferably made different for each of the second objects OJ2. This can reduce a frequency of the second objects OJ2 completely overlapping with each other in a case where the first object OJ1 includes a plurality of second objects OJ2.

A degree of wobbling of the outer edge OE of the second object OJ2 differs in accordance with the wind speed detected by the wind speed sensor 3. The degree of wobbling being different indicates at least one of that the predetermined period with which the shape of the outer edge OE returns to the original shape is different or that a size of concavo-convex portion of the outer edge OE is different.

In a case where the predetermined period is made different, the shape of the outer edge OE of the second object OJ2 returns to the original shape with a shorter period as the wind speed detected by the wind speed sensor 3 is higher.

In a case where the size of the concavo-convex portion is made different, the size of the concavo-convex portion of the outer edge OE of the second object OJ2 is greater as the wind speed detected by the wind speed sensor 3 is higher.

Returning to the description in FIG. 6, a fourth object OJ4, and a fifth object OJ5 are displayed in an overlapped manner on the spatial image G1. The fourth object OJ4 is an object indicating an air current and is an image indicating the air current with lines. The fifth object OJ5 is an object indicating an air-purifying substance and is an image indicating the air-purifying substance with dots. Note that in FIG. 6, each of lines extending from the indoor unit 4 and the outlet IH indicated in the spatial image G1 is the fourth object OJ4. Further, in FIG. 6, each of dots existing within the target space SP indicted in the spatial image G1 is the fifth object OJ5.

The fourth object OJ4 is displayed in association with the indoor unit 4 indicated in the spatial image G1. The fourth object OJ4 is displayed while avoiding overlapping with the first object OJ1, so that, for example, an overlapping area becomes equal to or less than a predetermined value.

A length of the fourth object OJ4 extending from the indoor unit 4 is set at a length in accordance with the air volume to be supplied to the target space SP by the indoor unit 4. In other words, the length of the fourth object OJ4 extending from the indoor unit 4 is set longer as the air volume to be supplied by the indoor unit 4 is greater. Further, a direction in which the fourth object OJ4 extends from the indoor unit 4 corresponds to a wind direction of the indoor unit 4.

The length of the fourth object OJ4 extending from the outlet IH is set at a length in accordance with the air volume at which hypochlorous acid is discharged from the outlet IH. In other words, the length of the fourth object OJ4 extending from the outlet IH is set longer as the air volume at which hypochlorous acid is discharged from the outlet IH is greater.

The fourth object OJ4 is displayed so as to extend from the indoor unit 4 or the outlet IH indicated in the spatial image G1 as time passes and disappears if the fourth object OJ4 extends to the set length.

The number of the fourth objects OJ4 per unit time extending from the indoor unit 4 may be set at the number in accordance with the air volume to be supplied to the target space SP by the indoor unit 4. Further, the number of the fourth objects OJ4 per unit time extending from the outlet IH indicated in the spatial image G1 may be set at the number in accordance with the air volume to be supplied to the target space SP from the outlet IH.

The fifth object OJ5 is displayed so as to be discharged from the indoor unit 4 or the outlet IH indicated in the spatial image G1. Further, the fifth object OJ5 is displayed so as to spread inside the target space SP indicated in the spatial image G1 as time passes after the fifth object OJ5 is discharged from the indoor unit 4 or the outlet IH indicated in the spatial image G1. A manner of spreading of the fifth object OJ5 is determined through test or simulation performed in advance.

The number of the fifth object OJ5 per unit time to be discharged from the indoor unit 4 increases as the air volume to be supplied to the target space SP by the indoor unit 4 is greater. Further, the number of the fifth object OJ5 per unit time to be discharged from the outlet IH increases as the air volume to be supplied to the target space SP from the outlet IH is greater.

A color of the fifth object OJ5 to be discharged from the indoor unit 4 is set to a color different from a color of the fifth object OJ5 to be discharged from the outlet IH. For example, the color of the fifth object OJ5 corresponding to particulate ions to be discharged from the indoor unit 4 is set to light blue, and the color of the fifth object OJ5 corresponding to hypochlorous acid to be discharged from the outlet IH is set to yellow. This allows the user P to grasp a manner of spreading of air-purifying substances for each type of the air-purifying substance.

As described above, the selection region SA is a region for selecting objects to be displayed in the first region A1 and includes seven items from the first to the seventh items.

In a case where the first item is not selected in the selection region SA, the fourth object OJ4 is not displayed in the spatial image G1. On the other hand, in a case where the first item is selected in the selection region SA, the fourth object OJ4 is displayed in the spatial image G1.

Further, in a case where the second item is not selected in the selection region SA, the fifth object OJ5 is not displayed in the spatial image G1. On the other hand, in a case where the second item is selected in the selection region SA, the fifth object OJ5 is displayed in the spatial image G1.

Returning to the description of FIG. 5, the second region A2 is a region for displaying the area image G2. The area image G2 is a schematic image of the target space SP viewed from above and is an image indicating each of the areas ERs provided in the target space SP. The area image G2 includes an area-specific image G21 for each area ER. The area image G2 illustrated in FIG. 5 includes six area images G2 of area-specific images G21-1, G21-2, G21-3, G21-4, G21-5, and G21-6. The area-specific image G21-1 indicates the area ER1, the area-specific image G21-2 indicates the area ER2, the area-specific image G21-3 indicates the area ER3, the area-specific image G21-4 indicates the area ER4, the area-specific image G21-5 indicates the area ER5, and the area-specific image G21-6 indicates the area ER6.

In the second region A2, each of the area-specific images G21 is displayed in a selectable manner. In other words, in the area image G2 to be displayed in the second region A2, the areas ER are displayed in a selectable manner. If one area ER is selected in the second region A2, in the third region A3, detection values of the air quality sensor 2 provided in the selected area ER are displayed for each type of air quality.

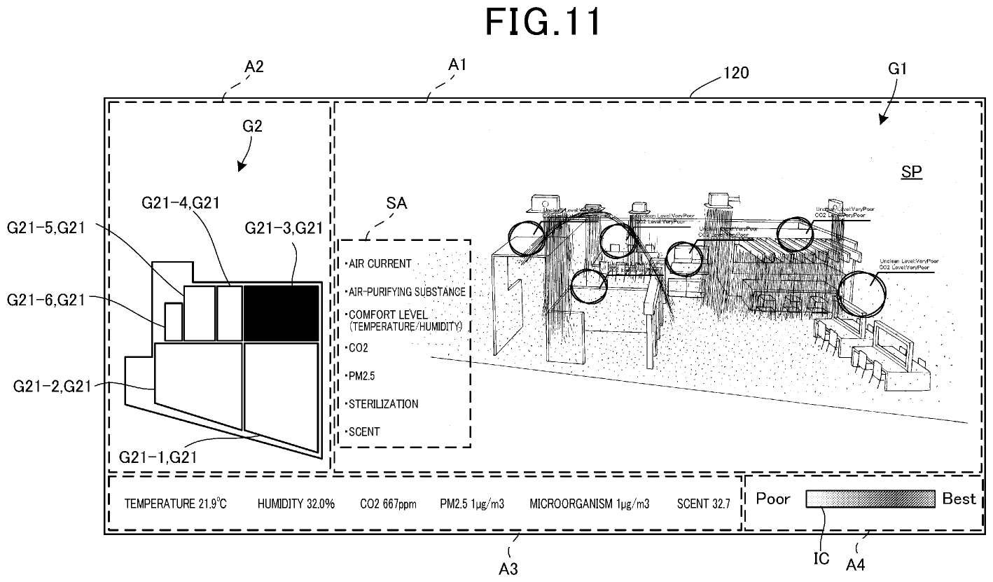

FIG. 11 is a view illustrating one example of the application UI 120. FIG. 11 illustrates the application UI 120 in a case where the area-specific image G21-3 is selected.

As described above, in the third region A3, the detection values of the air quality sensor 2 provided in the area ER selected in the second region A2 are displayed for each type of air quality. FIG. 11 illustrates a case where the area-specific image G21-3 is selected. Thus, in the third region A3 in FIG. 11, the detection values of the air quality sensor 2 provided in the area ER3 are displayed for each type of air quality.

Returning to the description of FIG. 5, in the fourth region A4, an indicator IC indicating evaluation indicated by the colors of the second objects OJ2 is displayed. This enables the user P to more clearly grasp the states of the air quality indicated by the second objects OJ2.

### [1-2. Operation]

Operation of the respective units of the display system 1000 will be described next.

First, the operation of the display system 1000 related to start of display of the application UI 120 will be described.

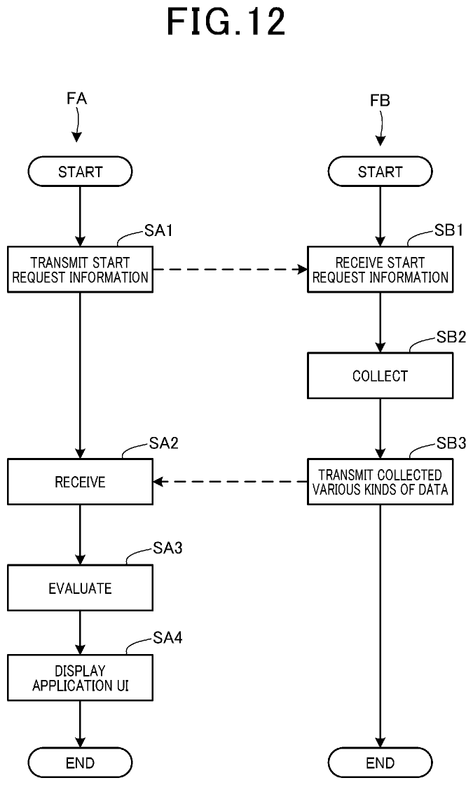

FIG. 12 is a flowchart indicating the operation of the display system 1000.

In FIG. 12, a flowchart FA indicates operation of the terminal device 1, and a flowchart FB indicates operation of the server device 6.

As indicated in the flowchart FA, the terminal communication control unit 101 transmits start request information to the server device 6 (step SA1). Processing in step SA1 is performed in a case where an instruction to start display of the application UI 120 is issued from the user P such as a case where the display application 111 is started.

The start request information is information for requesting various kinds of data related to display of the application UI 120.

The request information includes a space ID of the target space S. Note that the space ID is input to the terminal device 1 at a predetermined timing.

As indicated in the flowchart FB, the server communication control unit 601 receives the start request information (step SB1).

Then, the server communication control unit 601 collects various kinds of data to be transmitted to the terminal device 1 (step SB2).

Step SB2 will be described in detail.

In step SB2, the server communication control unit 601 specifies the record R1 with the space ID included in the start request information from the first management DB 612 and collects the specified record R1 as the various kinds of data to be transmitted to the terminal device 1.

Further, in step SAB2, the server communication control unit 601 specifies the record R2 with the space ID included in the start request information from the second management DB 613 and collects the specified record R2 as the various kinds of data to be transmitted to the terminal device 1.

Then, the server communication control unit 601 transmits the various kinds of data collected in step SB2 to the terminal device 1 (step SB3).

As indicated in the flowchart FA, the terminal communication control unit 101 receives the various kinds of data collected in step SB2 from the server device 6 (step SA2).

Then, the evaluation unit 102 evaluates the states of the air quality detected by the air quality sensor 2 for each of the air quality sensors 2 (step SA3).

Step SA3 is one example of an “evaluation step”.

In step SA3, the evaluation unit 102 performs the following processing for each of the air quality sensors 2, that is, for each piece of the first detection data D1 of the record R1 received in step SA2. In other words, the evaluation unit 102 evaluates the detection values of the air quality indicated by the first detection data D1 for each type of air quality.

Then, the display control unit 103 displays the application UI 120 using the touch panel 12 (step SA4).

Step SA4 is one example of a “display step”.

The processing in step SA4 will be described in detail.

In step SA4, the display control unit 103 acquires the evaluation results in step SA3 from the evaluation unit 102. More specifically, the display control unit 103 acquires the evaluation results for each type of air quality for each piece of the first detection data D1 of the record R1 received in step SA2.

In step SA4, the display control unit 103 displays the spatial image G1 indicated by the spatial image data of the record R1 received in step SA2 in the first region A1.

Further, in step SA4, the display control unit 103 displays the first object OJ1 in the spatial image G1 displayed in the first region A1.

Here, display of the first object OJ1 will be described.

The display control unit 103 generates the second objects OJ2 upon display of the first object OJ1 and determines a degree of wobbling of the outer edges OE.

The display control unit 103 generates the second objects OJ2 for each type of air quality for each of the air quality sensors 2, that is, for each piece of the first detection data D1 received in step SA2 on the basis of the acquired evaluation results.

In generation of the second objects OJ2, the display control unit 103 generates the second objects OJ2 in colors in accordance with the evaluation results by the evaluation unit 102. Note that in generation of the second object OJ2 of the third item, the display control unit 103 generates the second object OJ2 having the third objects OJ3 of the number in accordance with the detection value of the PM2.5 concentration indicated by the first detection data D1. Further, in generation of the second object OJ2 of the fourth item, the display control unit 103 generates the second object OJ2 having the third objects OJ3 of the number in accordance with the detection value of the microorganism concentration indicated by the first detection data D1.

The display control unit 103 determines the degree of wobbling of the outer edges OE for each of the air quality sensors 2, that is, for each piece of the first detection data D1 received in step SA2.

The display control unit 103 specifies the second detection data D2 corresponding to the first detection data D1 for each piece of the first detection data D1 with reference to the second management data received in step SA2. Then, the display control unit 103 determines the degree of wobbling of the outer edges OE of the second objects OJ2 to be a degree in accordance with the wind speed indicated by the specified second detection data D2 for each piece of the first detection data D1.

When the display control unit 103 generates the second objects OJ2 and determines the degree of wobbling of the outer edges OE for each of the air quality sensors 2, that is, for each piece of the first detection data D1 received in step SA2, the display control unit 103 displays the first object OJ1 in an overlapped manner on the spatial image G1.

The display control unit 103 recognizes positions corresponding to respective pieces of the first detection data D1 received in step SA2 in the spatial image G1 with reference to the second management data and the record R1 received in step SA2. Then, the display control unit 103 displays the first object OJ1 for each of the air quality sensors 2, that is, for each piece of the first detection data D1 received in step SA2 so as to achieve the recognized correspondence relationship. Note that the display control unit 103 wobbles the generated second objects OJ2 with the determined degree of wobbling of the outer edges OE and changes colors of the overlapping region TA1 and the non-overlapping region TA2 in display of the first object OJ1.

Returning to the description of display of the first region A1, in step SA4, the display control unit 103 displays the evaluation information HJ.

Here, display of the evaluation information HJ will be described.

The display control unit 103 generates the evaluation information HJ for each type of air quality for each of the air quality sensors 2, that is, for each piece of the first detection data D1 received in step SA2 on the basis of the evaluation results acquired from the evaluation unit 102. Then, the display control unit 103 recognizes positions corresponding to respective pieces of the first detection data D1 received in step SA2 in the spatial image G1 in a similar manner to the display of the first object OJ1. Then, the display control unit 103 displays the generated evaluation information HJ in the spatial image G1 for each piece of the first detection data D1 received in step SA2 so as to achieve the recognized correspondence relationship.

Returning to the description of the display of the first region A1, in step SA4, the display control unit 103 displays the fourth object OJ4 and the fifth object OJ5 in an overlapped manner on the spatial image G1.

The display control unit 103 recognizes positions corresponding to respective pieces of the first status data D3 in the spatial image G1 with reference to the second management data and the record R1 received in step SA2. Then, the display control unit 103 displays the fourth object OJ4 in accordance with the air volume and the wind direction of the indoor unit 4 indicated by the first status data D3 for each piece of the first status data D3 so as to achieve the recognized correspondence relationship. Here, a display position of the first object OJ1 is determined so as not to overlap with the fourth object OJ4.

The display control unit 103 displays the fifth object OJ5 from the outlet IH indicated in the spatial image G1 in accordance with the air volume indicated by the second status data D4 with reference to the second management data and the record R1 received in step SA2. Then, the display control unit 103 spreads the fifth object OJ5 in the spatial image G1 as time passes.

In step SA4, the display control unit 103 displays the area image G2 indicated by the area image data received in step SA2, in the second region A2.

Further, in step SA4, the display control unit 103 displays the indicator IC in the fourth region A4. Note that image data of the indicator IC may be recorded in the terminal memory 110 when the display application 111 is installed or may be received from the server device 6 along with the various kinds of data in step SA2.

As a result of this processing in step SA4, the display control unit 103 can display the application UI 120 as illustrated in FIG. 5.

Operation of the display system 1000 related to updating of display content of the application UI 120 will be described next.

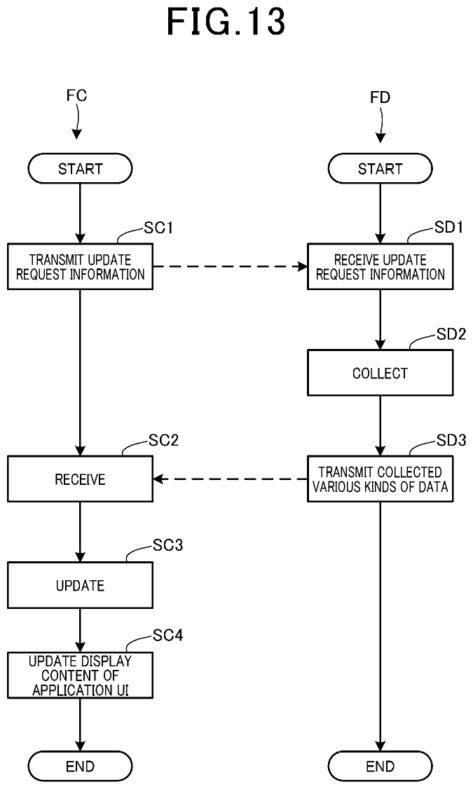

FIG. 13 is a flowchart indicating the operation of the display system 1000.

In FIG. 13, a flowchart FC indicates the operation of the terminal device 1, and a flowchart FD indicates the operation of the server device 6.

As indicated in the flowchart FC, the terminal communication control unit 101 transmits update request information to the server device 6 (step SC1). The processing in step SC1 is performed in a case where a predetermined period has elapsed since display of the application UI 120 was started, in a case where a predetermined period has elapsed since display content of the application UI 120 was updated last time, or the like.

The update request information is information for requesting update of the display content of the application UI 120. The update request information includes the space ID of the target space SP.

As indicated in the flowchart FD, the server communication control unit 601 receives the update request information (step SD1).

Then, the server communication control unit 601 collects various kinds of data to be transmitted to the terminal device 1 (step SD2).

Step SD2 will be described in detail.

In step SD2, the server communication control unit 601 specifies the record R1 with the space ID included in the update request information from the first management DB 612 and collects the specified record R1 as the various kinds of data to be transmitted to the terminal device 1.

Further, in step SD2, the server communication control unit 601 specifies the record R2 with the space ID included in the start request information from the second management DB 613 and collects the specified record R2 as the various kinds of data to be transmitted to the terminal device 1.

Then, the server communication control unit 601 transmits the various kinds of data collected in step SD2 to the terminal device 1 (step SD3).

As indicated in the flowchart FC, the terminal communication control unit 101 receives the various kinds of data collected in step SD2 from the server device 6 (step SC2).

Then, the evaluation unit 102 evaluates the states of air quality detected by the air quality sensor 2 for each of the air quality sensors 2 (step SC3). In step SC3, the evaluation unit 102 performs evaluation in a similar manner to step SA3.

Step SC3 is one example of an “evaluation step”.

Then, the display control unit 103 displays the application UI 120 using the touch panel 12 (step SC4). In step SC4, the display control unit 103 generates each object and the evaluation information HJ in a similar manner to step SA4 and displays them in the first region A1.

Step SC4 is one example of a “display step”.

The operation of the terminal device 1 while the application UI 120 is displayed will be described next.

First, the operation of the terminal device 1 related to selection of an item in the selection region SA will be described.

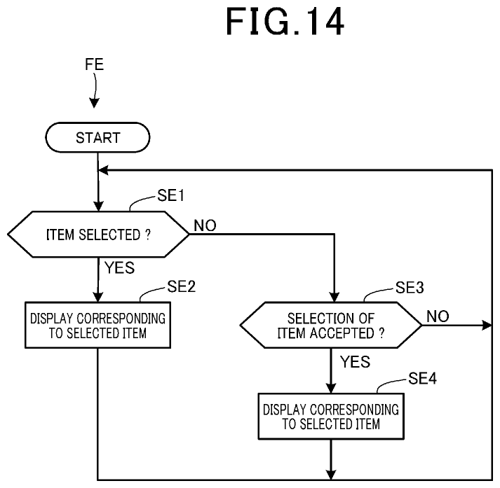

FIG. 14 is a flowchart FE indicating the operation of the terminal device 1. The processing in FIG. 14 is repeatedly performed while the application UI 120 is displayed.

The display control unit 103 determines whether or not an item is selected in the selectin region SA (step SE1). In other words, the display control unit 103 determines whether or not the acceptance unit 104 accepts selection of an item in step SE1.

In a case where it is determined that an item is selected in the selection region SA (step SE1: Yes), the display control unit 103 displays an object corresponding to the item selected in the selection region SA (step SE2).

In step SE2, in a case where at least one of the third to the seventh items is selected in the selection region SA, the display control unit 103 displays the evaluation information HJ corresponding to the selected item.

Returning to the description of step SE1, in a case where it is determined that an item is not selected in the selection region SA (step SE1: No), the display control unit 103 determines whether or not the acceptance unit 104 accepts selection of an item in the selection region SA (step SE3).

In a case where it is determined that the acceptance unit 104 does not accept selection of an item (step SE3: No), the process returns to step SA1, and the display control unit 103 performs processing in step SA1 and subsequent processing again.

On the other hand, in a case where it is determined that the acceptance unit 104 accepts selection of an item (step SE3: Yes), the display control unit 103 displays an object corresponding to the selected item (step SE4).

In a case where at least one of the third to the seventh items is selected in the selection region SA, the display control unit 103 displays the evaluation information HJ corresponding to the selected item in step SE4.

The operation of the terminal device 1 related to selection of the area ER will be described next.

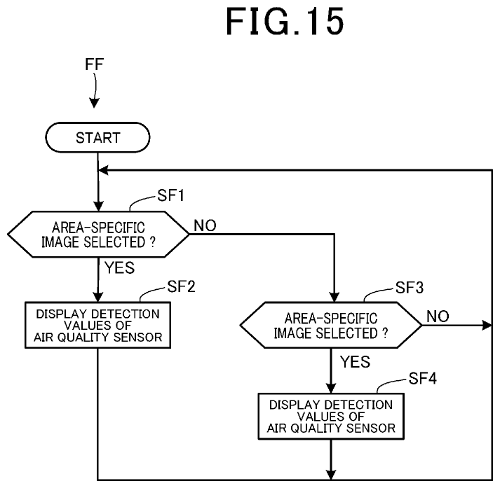

FIG. 15 is a flowchart FF indicating the operation of the terminal device 1. The processing in FIG. 15 is repeatedly performed independently of FIG. 14 while the application UI 120 is displayed.

The display control unit 103 determines whether or not the area-specific image G21 is selected (step SF1). In other words, in step SF1, the display control unit 103 determines whether or not the acceptance unit 104 accepts selection of the area ER in the area image G2.

In a case where it is determined that the area-specific image G21 is selected (step SF1: Yes), the display control unit 103 displays detection values of the air quality sensor 2 provided in the area ER corresponding to the selected area-specific image G21 in the third region A3 (step SF2).

Returning to the description of step SF1, in a case where it is determined that the area-specific image G21 is not selected (step SF1: No), the display control unit 103 determines whether or not the area-specific image G21 is selected (step SF3). In other words, in step SF1, the display control unit 103 determines whether or not the acceptance unit 104 accepts selection of the area ER in the area image G2.

In a case where it is determined that the area-specific image G21 is not selected (step SF3: No), the processing returns to step SF1, and the display control unit 103 performs the processing in step SF1 and subsequent processing again.

On the other hand, in a case where it is determined that the area-specific image G21 is selected (step SF3: Yes), the display control unit 103 displays detection values of the air quality sensor 2 corresponding to the selected area-specific image G21 (step SF4). In step SF4, the display control unit 103 recognizes the first sensor ID of the air quality sensor 2 provided in the selected area ER with reference to the received first management data. Then, the display control unit 103 specifies the first detection data D1 corresponding to the first sensor ID recognized from the received record R1 and displays the detection values of the air quality sensor 2 indicated by the specified first detection data D1 in the third region A3.

### [1-3. Effects, and the Like]

As described above, a display method by the display system 1000 includes an evaluation step of evaluating states of air quality for each type of air quality on the basis of detection values of the air quality sensor 2 that detects the states of the air quality of the target space SP. Further, the display method includes a display step of displaying by the terminal device 1, the first object OJ1 indicating the states of the air quality in the spatial image G1 indicating the target space SP on the basis of evaluation results in the evaluation step. The first object OJ1 is a combination of a plurality of second objects OJ2 indicating the states of the air quality for each type of air quality. In the display step, the second objects OJ2 are displayed in colors corresponding to the evaluation results in the evaluation step for each of the second objects OJ2 while display manner is changed for each of the second objects OJ2.

According to this, by displaying the plurality of second objects OJ2 in colors corresponding to the evaluation results, it is possible to allow a user to easily grasp impression of an overall state of the air quality based on states of a plurality of types of air quality. Further, by displaying the second objects OJ2 while changing display manner, it is possible to clearly specify that the first object OJ1 is not part of the target space SP indicated by the spatial image G1. This makes it possible to allow the user to easily grasp that the first object OJ1 is an object indicating states of air quality. It is therefore possible to allow the user P to easily grasp states of a plurality of types of air quality and visually confirm an overall state of air quality based on the states of the plurality of types of air quality.

In the display step, the second objects OJ2 are displayed while at least part of the second objects OJ2 are made to overlap with each other.

According to this, compared to a case where the second objects OJ2 are not made to overlap with each other, the states of a plurality of types of air quality can be displayed in a narrow range. This makes it possible to allow the user to grasp the states of the plurality of types of air quality more easily compared to a case where the second objects OJ2 are not made to overlap with each other.

In the display step, the second objects OJ2 are displayed while the outer edges OE of the second objects OJ2 are made to wobble.

According to this, the first object OJ1 can be displayed as an object that mimics air, so that it is possible to allow the user to easily grasp that the first object OJ1 indicates states of air quality. It is therefore possible to allow the user to grasp states of a plurality of types of air quality more easily. Further, a portion where the second objects OJ2 overlap with each other can be changed with time, and thus, the user can more easily visually confirm respective evaluation results of the states of air quality. It is therefore possible to allow the user to easily visually confirm an overall state of air quality based on states of a plurality of types of air quality.

A degree of wobbling of the outer edges OE of the second objects OJ2 is a degree in accordance with wind speed around a position where the air quality sensor 2 is provided.

According to this, it is possible to allow the user to grasp states of air quality and grasp wind speed around the first object OJ1.

In the display step, in a case where the detection values of the air quality sensor 2 include a detection value equal to or greater than a threshold, the alert AR is displayed in association with the first object OJ1.

According to this, it is possible to make the user grasp that the user should pay attention to the states of air quality.

The air quality sensor 2 detects concentration of an air-polluting substance that pollutes air of the target space SP. In the display step, the third objects OJ3 indicating the air-polluting substance are displayed in the second objects OJ2, the number of the third objects OJ3 corresponding to the concentration of the air-polluting substance detected by the air quality sensor 2.

According to this, it is possible to make the user easily grasp the concentration of the air-polluting substance that pollutes air of the target space SP.

The air quality sensor 2 is provided in each of a plurality of areas ER within the target space SP. In the display step, the first object OJ1 is displayed in each of the plurality of areas ER within the target space SP indicated by the spatial image G1.

According to this, it is possible to allow the user P to easily grasp states of a plurality of types of air quality for each area ER and allow the user P to visually confirm an overall state of air quality based on the states of the plurality of types of air quality for each area ER.

In the display step, the area image G2 indicating a plurality of areas ER is displayed. In the display step, in a case where one of the plurality of areas ER is selected in the area image G2, detection values of the air quality sensor 2 provided in the selected area ER are displayed for each type of air quality.

According to this, the detection values of the air quality sensor 2 are displayed for each type of air quality, so that it is possible to allow the user to specifically grasp the detection values of the air quality sensor 2 provided in the selected area ER for each type of air quality.

The terminal device 1 includes the display control unit 103 that displays the first object OJ1 indicating states of air quality in the spatial image G1 indicating the target space SP using the touch panel 12 on the basis of evaluation results by the evaluation unit 102 that evaluates the states of the air quality for each type of air quality on the basis of detection values of the air quality sensor 2 that detects states of air quality of the target space SP. The first object OJ1 is a combination of a plurality of second objects OJ2 indicating the states of the air quality for each type of air quality. The display control unit 103 displays the second objects OJ2 in colors corresponding to the evaluation results by the evaluation unit 102 for each of the second objects OJ2 while changing display manner for each of the second objects OJ2.

According to this, effects similar to those in the above-described display method are provided.

The display system 1000 includes the touch panel 12, the evaluation unit 102 that evaluates states of air quality for each type of the air quality on the basis of detection values of the air quality sensor 2 that detects the states of the air quality of the target space SP, and the display control unit 103 that displays the first object OJ1 indicating the states of the air quality in the spatial image G1 indicating the target space SP using the touch panel 12 on the basis of the evaluation results by the evaluation unit 102. The first object OJ1 is a combination of a plurality of second objects indicating the states of the air quality for each type of air quality. The display control unit 103 displays the second objects OJ2 in colors corresponding to the evaluation results by the evaluation unit 102 for each of the second objects OJ2 while changing display manner for each of the second objects OJ2.

According to this, effects similar to those in the above-described display method are provided.

The display application 111 causes the terminal processor 100 of the terminal device 1 to function as the display control unit 103 that displays the first object OJ1 indicating states of air quality in the spatial image G1 indicating the target space SP using the touch panel 12 on the basis of evaluation results by the evaluation unit 102 that evaluates the states of the air quality for each type of air quality on the basis of detection values of the air quality sensor 2 that detects the states of the air quality of the target space SP. The first object OJ1 is a combination of a plurality of second objects OJ2 indicating the states of the air quality for each type of air quality. The display control unit 103 displays the second objects OJ2 in colors corresponding to the evaluation results by the evaluation unit 102 for each of the second objects OJ2 while changing display manner for each of the second objects OJ2.

According to this, effects similar to those in the above-described display method are provided.

### Second Embodiment

A second embodiment will be described next.

In description of the second embodiment, the same reference numerals will be assigned to components that are the same as the components of respective units of the display system 1000 in the first embodiment, and detailed description will be omitted as appropriate.

When the first embodiment and the second embodiment are compared, the second embodiment is different from the first embodiment in display content of the third region A3.

### [2-1. Configuration]

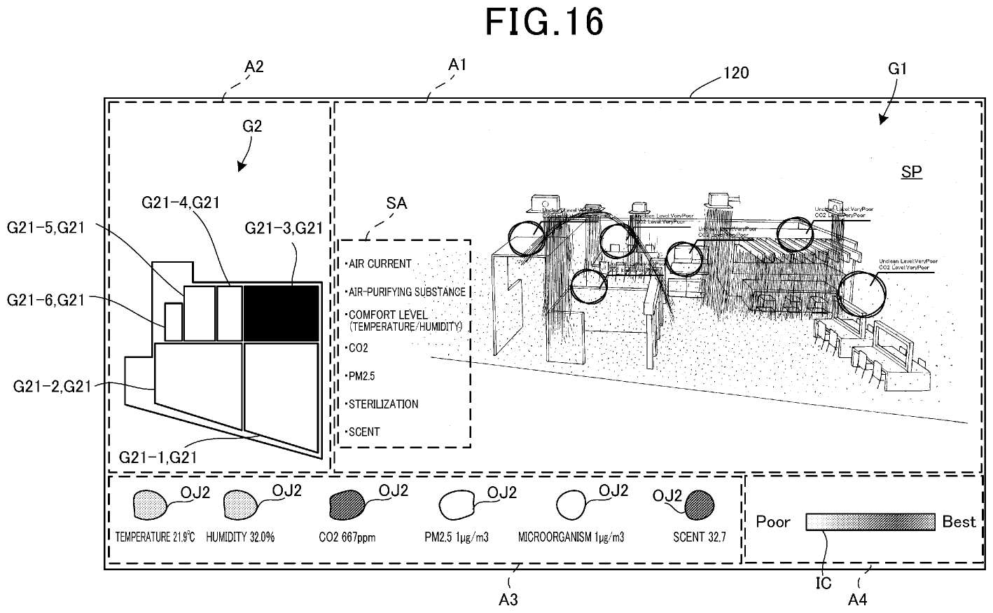

FIG. 16 is a view illustrating one example of the application UI 120 according to the second embodiment.

FIG. 16 illustrates the application UI 120 in a case where the area-specific image G21-3 is selected.

The display control unit 103 of the second embodiment displays the second objects OJ2 in which types of air quality respectively correspond to detection values of the air quality sensor 2 in the third region A3.

The second objects OJ2 displayed in the third region A3 are colored in accordance with the evaluation results in a similar manner to the second objects OJ2 displayed in the first region A1. Further, the second objects OJ2 displayed in the third region A3 are displayed while the outer edges OE wobble in a similar manner to the second objects OJ2 displayed in the first region A1.

### [2-3. Effects]

The second embodiment 2 provides effects similar to those of the first embodiment.

Further, in the display step of the second embodiment, the second objects OJ2 corresponding to types of air quality are displayed in association with the detection values of the air quality sensor 2 for each type of air quality.

According to this, the second objects OJ2 corresponding to types of air quality are displayed in association with the detection values of the air quality sensor 2. It is therefore possible to allow the user to grasp what kinds of second objects OJ2 are displayed and values of the detection values for each type of air quality. Thus, the user can easily and specifically grasp the states of air quality indicated by the first object OJ1, so that it is possible to allow the user to easily and specifically grasp states of a plurality of types of air quality.

### OTHER EMBODIMENTS

As described above, the first embodiment and the second embodiment have been described as examples disclosed in the present application. However, the technique in the present disclosure is not limited to these and can be applied to embodiments in which change, replacement, addition, omission, and the like, are made. Further, a new embodiment can be obtained by combining the components described in the first and second embodiments described above. Thus, other embodiments will be described below.

In the above-described embodiments, the degree of wobbling of the outer edges OE of the second object OJ2 is a degree in accordance with wind speed around a position where the air quality sensor 2 is provided. In another embodiment, the degree of wobbling of the outer edges OE of the second object OJ2 may be made a degree in accordance with outdoor wind speed, that is, wind speed around the building H. In the other embodiment, for example, the display control unit 103 acquires weather data from a predetermined database and estimates outdoor wind speed on the basis of the acquired weather data. Then, the display control unit 103 determines the degree of wobbling of the outer edges OE of the second object OJ2 in accordance with the estimated wind speed.

According to the other embodiment, it is possible to allow the user to grasp the states of air quality and grasp outdoor wind speed. This enables comparison between indoor and outdoor.

In the above-described embodiments, the wind speed around the position where the air quality sensor 2 is provided is detected by the wind speed sensor 3. In another embodiment, the wind speed around the position where the air quality sensor 2 is provided may be estimated on the basis of the air volume to be supplied by the indoor unit 4 located closest to the air quality sensor 2.

While in the above-described embodiments, hypochlorous acid and particulate ions are exemplified as the substance that purify air of the target space SP, the substance is not limited to these and may be, for example, ozone water, or the like.

In the above-described embodiments, the evaluation information HJ includes names of items and evaluation results. In another embodiment, the evaluation information HJ does not have to include names of items.

In the above-described embodiments, PM2.5 and microorganisms are exemplified as the air-polluting substance. In another embodiment, the air-polluting substance may include a gas that pollutes air such as CO2 and a substance that pollutes air, other than PM2.5 and microorganisms. In the other embodiment, for example, the second object OJ2 of the fourth item includes the third objects OJ3 indicating CO2, of the number in accordance with CO2 concentration.

In the above-described embodiments, the shapes of the second objects OJ2 in a case where the outer edges OE are not made to wobble are the same regardless of types of air quality. In another embodiment, the shapes of the second objects OJ2 may be different for each type of air quality. For example, in the other embodiment, the shape of the second object OJ2 of the third item may be made a circular shape, the shape of the second object OJ2 of the fourth item may be made a triangular shape, the shape of the second object OJ2 of the fifth item may be made a quadrangular shape, the shape of the second object OJ2 of the sixth item may be made a star shape, and the shape of the second object OJ2 of the seventh item may be made a pentagonal shape.

According to the other embodiment, it is possible to allow the user to grasp that types of indicated air quality are different for each of the second objects OJ2, so that it is possible to allow the user to grasp states of a plurality of types of air quality more easily.

In the above-described embodiments, a case has been described as an example where types of air quality to be detected by the air quality sensor 2 are six types. However, the types of air quality to be detected by the air quality sensor 2 are not limited to six types and may be five types or less or seven types or more, and other types such as concentration of pollen may be included in place of at least one of the six types indicated in the embodiments.

In the above-described embodiments, in a case where the first object OJ1 includes a plurality of second objects OJ2, the second objects OJ2 overlap with each other. In another embodiment, in that case, the second objects OJ2 do not have to be made to overlap with each other.

While in the above-described embodiments, descriptor has been made in a manner that the outer edges OE are made to wobble as an example of changing the display manner of the second objects OJ2, the display manner of the second objects OJ2 may be changed in a different manner, and, for example, the second objects OJ2 themselves may be made to wobble.

In the above-described embodiments, one air quality sensor 2 detects a plurality of types of air quality. In another embodiment, the display system 1000 may include the air quality sensor 2 that detects a state of one type of air quality for each type of air quality to be detected.

In the above-described embodiments, the color of the overlapping region TA1 is made the mixed color of the second objects OJ2 that overlap with each other. In another embodiment, the color of the overlapping region TA1 may be made a color of the second object OJ2 with the lowest evaluation result among the overlapping second objects OJ2.

In the above-described embodiments, a case has been described as an example where the target space SP is divided into six areas ER. In another embodiment, the number of areas ER obtained by dividing the target space SP may be five or less or seven or more.

While in the above-described embodiments, one air quality sensor 2 is provided in one area ER, the number of the air quality sensors 2 provided in one area ER is not limited to one, and a plurality of air quality sensors 2 may be provided.

While in the above-described embodiments, at least one of the indoor unit 4 or the outlet IH is provided in accordance with the area ER, both the indoor unit 4 and the outlet IH may be provided in the area ER.

In the above-described embodiments, a smartphone and a tablet computer are described as examples of the terminal device 1. In another embodiment, the terminal device 1 may be a desktop computer or a laptop computer. Further, in the other embodiment, the terminal device 1 does not have to include a display unit such as a display and a touch panel. In this case, the terminal device 1 causes the application UI 120 to be displayed in the display unit such as a display that is externally connected.

In the above-described embodiments, the server device 6 is described as an example of a device that stores the first management DB 612, the second management DB 613 and performs processing with reference to these management DBs. In another embodiment, a device that stores the first management DB 612 and the second management DB 613 and performs processing with reference to these management DBs does not have to be a device connected to the network NW and may be a management device that is connected to the local network of the building H and manages the target space SP.

While in the above-described embodiments, the terminal processor 100 functions as the evaluation unit 102, in another embodiment, the terminal processor 100 does not have to function as the evaluation unit 102. In the other embodiment, the server processor 600 functions as the evaluation unit 102. Further, in the other embodiment, the evaluation results of the evaluation unit 102 are transmitted from the server device 6 to the terminal device 1, and the terminal device 1 displays the application UI 120 on the basis of the received evaluation results.

In the above-described embodiments, the display application 111 is described as an example of the “program”. In another embodiment, the “program” may be a program included in an operation system of the terminal device 1 or may be a program that has already been downloaded.

The terminal processor 100 and the server processor 600 may be constituted with a single processor or may be constituted with a plurality of processors. These processors may be hardware programmed to implement corresponding functional units. In other words, these processors may be constituted with, for example, an application specific integrated circuit (ASIC) and a field programmable gate array (FPGA).

The configurations of the terminal device 1 and the server device 6 illustrated in FIG. 3 are one example, and specific implementation forms are not particularly limited. In other words, hardware that individually corresponds to respective units is not necessarily mounted, and functions of the respective units may be implemented by one processor executing programs. Further, part of the functions to be implemented by software in the above-described embodiments may be implemented by hardware or part of the functions to be implemented by hardware may be implemented by software.

Steps of the operation indicated in FIG. 12 to FIG. 15 are obtained by dividing the operation in accordance with main processing content to facilitate understanding of the operation, and the operation is not limited by a way of division or a name of the processing unit. The operation may be divided into more steps in accordance with the processing content. Further, the operation may be divided so that one step includes more kinds of processing. Further, the order of the steps may be replaced as appropriate within a range not deviating from the gist of the present disclosure.

Note that the above-described embodiments are provided to exemplify the technique in the present disclosure, and thus, various changes, replacement, addition, omission, and the like, can be made in a range of the claims or equivalent range thereof.

### SUPPLEMENT

The following techniques are disclosed by the above description of the embodiments.

### (Technique 1)

A display method including an evaluation step of evaluating states of air quality for each type of the air quality on the basis of detection values of an air quality sensor that detects the states of the air quality of target space, and a display step of displaying by a terminal device, a first object indicating the states of the air quality in a spatial image indicating the target space on the basis of evaluation results in the evaluation step, in which the first object is a combination of a plurality of second objects indicating the states of the air quality for each type of the air quality, and in the display step, the second objects are displayed in colors corresponding to the evaluation results in the evaluation step for each of the second objects while display manner is changed for each of the second objects.

According to this, by displaying the plurality of second objects in colors corresponding to the evaluation results, it is possible to allow the user to easily grasp impression of an overall state of air quality based on states of a plurality of types of air quality. Further, by displaying the second objects while changing the display manner, it is possible to clearly specify that the first object is not part of the target space indicated by the spatial image. It is therefore possible to allow the user to easily grasp that the first object is an object that indicates the states of the air quality. This makes it possible to allow the user to easily grasp the states of the plurality of types of air quality and visually confirm an overall state of air quality based on the states of the plurality of types of air quality.

### (Technique 2)

The display method according to technique 1, in which in the display step, the second objects are displayed while at least part of the second objects are made to overlap with each other.

According to this, compared to a case where the second objects are not made to overlap with each other, the states of the plurality of types of air quality can be displayed in a narrow range. Thus, compared to a case where the second objects are not made to overlap with each other, it is possible to allow the user to grasp the states of the plurality of types of air quality more easily.

### (Technique 3)

The display method according to technique 1 or 2, in which in the display step, the second objects are displayed while outer edges of the second objects are made to wobble.

According to this, the first object can be displayed as an object that mimics air, so that it is possible to allow the user to easily grasp that the first object indicates states of air quality. It is therefore possible to allow the user to grasp the states of the plurality of types of air quality more easily.

### (Technique 4)

The display method according to technique 3, in which a degree of wobbling of the outer edges of the second objects is a degree in accordance with wind speed around a position where the air quality sensor is provided.

According to this, it is possible to allow the user to grasp the states of air quality and grasp the wind speed around the first object.

### (Technique 5)

The display method according to technique 3, in which the target space is indoor, and a degree of wobbling of the outer edges of the second objects is a degree in accordance with outdoor wind speed.

According to this, it is possible to allow the user to grasp the states of air quality and grasp the outdoor wind speed. This enables comparison between indoor and outdoor.

### (Technique 6)

The display method according to any one of technique 1 to technique 5, in which in the display step, in a case where the detection values of the air quality sensor include a detection value equal to or greater than a threshold, an alert is displayed in association with the first object.

According to this, it is possible to allow the user to grasp that the user should pay attention to the states of air quality.

### (Technique 7)

The display method according to any one of technique 1 to technique 6, in which the air quality sensor detects concentration of an air-polluting substance that pollutes air of the target space, and in the display step, third objects indicating the air-polluting substance are displayed in the second objects, the number of the third objects corresponding to the concentration of the air-polluting substance detected by the air quality sensor.

According to this, it is possible to allow the user to easily grasp the concentration of the air-polluting substance that pollutes air of the target space.

### (Technique 8)

The display method according to any one of technique 1 to technique 7, in which the air quality sensor is provided in each of a plurality of areas within the target space, and in the display step, the first object is displayed in each of the plurality of areas within the target space indicated by the spatial image.

According to this, it is possible to allow the user to easily grasp the states of the plurality of types of air quality for each area and allow the user to visually confirm an overall state of air quality based on the states of the plurality of types of air quality for each area.

### (Technique 9)

The display method according to technique 8, in which in the display step, an area image indicating the plurality of areas is displayed, and in a case where one of the plurality of areas is selected in the area image, the detection values of the air quality sensor provided in the selected area are displayed for each type of the air quality.

According to this, the detection values of the air quality sensor are displayed for each type of air quality, so that it is possible to allow the user to specifically grasp the detection values of the air quality sensor provided in the selected area for each type of air quality.

### (Technique 10)

The display method according to technique 9, in which in the display step, the second objects corresponding to types of the air quality are displayed in association with the detection values of the air quality sensor for each type of the air quality.

According to this, the second objects corresponding to the types of the air quality are displayed in association with the detection values of the air quality sensor. It is therefore possible to allow the user to grasp what kinds of second objects are displayed and values of the detection values for each type of air quality. Thus, the user can easily and specifically grasp the states of the air quality indicated by the first object, so that it is possible to allow the user to easily and specifically grasp the states of the plurality of types of air quality.

### (Technique 11)

The display method according to any one of technique 1 to technique 10, in which shapes of the second objects are different for each type of the air quality.

According to this, it is possible to allow the user to easily grasp that the types of the indicated air quality are different for each of the second objects. It is therefore possible to allow the user to grasp the states of the plurality of types of air quality more easily.

### (Technique 12)

A terminal device including a display control unit that displays a first object indicating states of air quality in a spatial image indicating target space using a display unit on the basis of evaluation results by an evaluation unit that evaluates the states of the air quality for each type of the air quality on the basis of detection values of an air quality sensor that detects the states of the air quality of the target space, in which the first object is a combination of a plurality of second objects indicating the states of the air quality for each type of the air quality, and the display control unit displays the second objects in colors corresponding to the evaluation results by the evaluation unit for each of the second objects while changing display manner for each of the second objects.

According to this, effects similar to the effects of the display method of technique 1 can be provided.

### (Technique 13)

A display system including a display unit, an evaluation unit that evaluates states of air quality for each type of the air quality on the basis of detection values of an air quality sensor that detects the states of the air quality of target space, and a display control unit that displays a first object indicating the states of the air quality in a spatial image indicating the target space using the display unit on the basis of evaluation results by the evaluation unit, in which the first object is a combination of a plurality of second objects indicating the states of the air quality for each type of the air quality, and the display control unit displays the second objects in colors corresponding to the evaluation results by the evaluation unit for each of the second objects while changing display manner for each of the second objects.

According to this, effects similar to the effects of the display method of technique 1 can be provided.

### (Technique 14)

A non-transitory computer readable storage medium storing a program for causing a processor of a terminal device to function as a display control unit that displays a first object indicating states of air quality in a spatial image indicating target space using a display unit on the basis of evaluation results by an evaluation unit that evaluates the states of the air quality for each type of the air quality on the basis of detection values of an air quality sensor that detects the states of the air quality of the target space, in which the first object is a combination of a plurality of second objects indicating the states of the air quality for each type of the air quality, and the display control unit displays the second objects in colors corresponding to the evaluation results by the evaluation unit for each of the second objects while changing display manner for each of the second objects.

According to this, effects similar to the effects of the display method of technique 1 can be provided.

As described above, the display method, the terminal device, the display system and the storage medium according to the present invention can be utilized in applications for allowing a user to grasp states of air quality.
1 Terminal device
2 Air quality sensor
3 Wind speed sensor
4 Indoor unit
5 First air purification device
6 Server device
7 Second air purification device
10 Terminal control device
11 Terminal communication unit
12 Touch panel (display unit)
100 Terminal processor (processor)
101 Terminal communication control unit
102 Evaluation unit
103 Display control unit
104 Acceptance unit
110 Terminal memory
111 Display application (program)
1000 Display system
AR Alert
ER Area
G1 Spatial image
G2 Area image
OE Outer edge
OJ1 First object
OJ2 Second object
OJ3 Third object
OJ4 Fourth object
OJ5 Fifth object
SA3, SC3 Step (evaluation step)
SA4, SC4 Step (display step)
SP Target space

What is claimed is:

1. A display method comprising:
an evaluation step of evaluating states of air quality for each type of the air quality on a basis of detection values of an air quality sensor that detects the states of the air quality of target space; and
a display step of displaying by a terminal device, a first object indicating the states of the air quality in a spatial image indicating the target space on a basis of evaluation results in the evaluation step,
wherein the first object is a combination of a plurality of second objects indicating the states of the air quality for each type of the air quality, and
in the display step, the second objects are displayed in colors corresponding to the evaluation results in the evaluation step for each of the second objects while display manner is changed for each of the second objects.

2. The display method according to claim 1,
wherein in the display step, the second objects are displayed while at least part of the second objects are made to overlap with each other.

3. The display method according to claim 1,
wherein in the display step, the second objects are displayed while outer edges of the second objects are made to wobble.

4. The display method according to claim 3,
wherein a degree of wobbling of the outer edges of the second objects is a degree in accordance with wind speed around a position where the air quality sensor is provided.

5. The display method according to claim 3,
wherein the target space is indoor, and
a degree of wobbling of the outer edges of the second objects is a degree in accordance with outdoor wind speed.

6. The display method according to claim 1,
wherein in the display step, in a case where the detection values of the air quality sensor include a detection value equal to or greater than a threshold, an alert is displayed in association with the first object.

7. The display method according to claim 1,
wherein the air quality sensor detects concentration of an air-polluting substance that pollutes air of the target space, and
in the display step, third objects indicating the air-polluting substance are displayed in the second objects, the number of the third objects corresponding to the concentration of the air-polluting substance detected by the air quality sensor.

8. The display method according to claim 1,
wherein the air quality sensor is provided in each of a plurality of areas within the target space, and
in the display step, the first object is displayed in each of the plurality of areas within the target space indicated by the spatial image.

9. The display method according to claim 8,
wherein in the display step, an area image indicating the plurality of areas is displayed, and
in a case where one of the plurality of areas is selected in the area image, the detection values of the air quality sensor provided in the selected area are displayed for each type of the air quality.

10. The display method according to claim 9,
wherein in the display step, the second objects corresponding to types of the air quality are displayed in association with the detection values of the air quality sensor for each type of the air quality.

11. The display method according to claim 1,
wherein shapes of the second objects are different for each type of the air quality.

12. A terminal device comprising:
a display control unit that displays a first object indicating states of air quality in a spatial image indicating target space using a display unit on a basis of evaluation results by an evaluation unit that evaluates the states of the air quality for each type of the air quality on a basis of detection values of an air quality sensor that detects the states of the air quality of the target space,
wherein the first object is a combination of a plurality of second objects indicating the states of the air quality for each type of the air quality, and
the display control unit displays the second objects in colors corresponding to the evaluation results by the evaluation unit for each of the second objects while changing display manner for each of the second objects.

13. A display system comprising:
a display unit;
an evaluation unit that evaluates states of air quality for each type of the air quality on a basis of detection values of an air quality sensor that detects the states of the air quality of target space; and
a display control unit that displays a first object indicating the states of the air quality in a spatial image indicating the target space using the display unit on a basis of evaluation results by the evaluation unit,
wherein the first object is a combination of a plurality of second objects indicating the states of the air quality for each type of the air quality, and
the display control unit displays the second objects in colors corresponding to the evaluation results by the evaluation unit for each of the second objects while changing display manner for each of the second objects.

14. A non-transitory computer readable storage medium storing a program for causing a processor of a terminal device to function as:
a display control unit that displays a first object indicating states of air quality in a spatial image indicating target space using a display unit on a basis of evaluation results by an evaluation unit that evaluates the states of the air quality for each type of the air quality on a basis of detection values of an air quality sensor that detects the states of the air quality of the target space,
wherein the first object is a combination of a plurality of second objects indicating the states of the air quality for each type of the air quality, and
the display control unit displays the second objects in colors corresponding to the evaluation results by the evaluation unit for each of the second objects while changing display manner for each of the second objects.
